(19) United States
(12) Patent Application Publication
(10) Pub. No.: US 2006/0013870 A1
(43) Pub. Date: Jan. 19, 2006

(54) Pharmaceutical compositions of hops resins

(76) Inventor: Eric H. Kuhrts, Bodega, CA (US)

Correspondence Address: THORPE NORTH & WESTERN, LLP., 8180 SOUTH 700 EAST, SUITE 200, SANDY, UT, 84070, US

(21) Appl. No.: 11/152,023

(22) Filed: Jun. 13, 2005

Related U.S. Application Data
(63) Continuation-in-part of application No. 10/140,495, filed on May 6, 2002.

(51) Int. Cl. A61K 36/18; A61K 9/20

(52) U.S. Cl. 424/464; 424/778

(57) ABSTRACT

The present invention is drawn to a pharmaceutical composition comprising a dry free flowing powder. The powder can include various combinations of alpha acid, iso-alpha acids, and beta acids. The composition can further include a silica salt absorbent and/or an anti-oxidant. These compositions are preferably prepared by mixing hops extract with an absorbent in a high intensity mixer without added solvent.

The present application is a Continuation-In-Part Application of U.S. patent application Ser. No. 10/140,495, filed on May 6, 2002, which is incorporated herein by reference in its entirety.

### FIELD OF THE INVENTION

This invention relates to unique pharmaceutical powder compositions comprising extracts of hops resins. The compositions include high levels of active agents that can be extracted from hops flowers, and are in a form that does not cause nausea or other undesired side effects when administered orally.

### BACKGROUND OF THE INVENTION

Many botanical substances contain chemicals that have been found to be useful for the therapeutic treatment of various medical conditions. Since these chemicals are usually present in very small amounts, techniques have been developed to extract these substances to concentrate the therapeutically active agents. Various methods are available for extraction and purification of such substances, including the use of organic solvents, microwave systems, and supercritical CO₂ extraction. Organic solvent-based extractions utilize added solvents that are evaporated to form concentrated extract, which results in a damp, pasty mass that is typically further spray-dried onto a carrier for delivery. Alternatively, supercritical CO₂ extraction is another preferred method of collecting such extracts. With many botanicals, supercritical CO₂ extraction has certain benefits over organic solvent-based extractions. This extraction method yields a thick, high viscosity resin, oil, or other fluid-like material having a honey-like consistency, for example.

One pharmaceutically useful botanical substance is the extract of hops (Humulus lupulus L.). Hops cone flowers contain a variety of active agents, including alpha acids, iso-alpha acids, and beta acids, as well as a number of flavonoids and essential oils. Humulone, one of the alpha acids found in hops, has been demonstrated to suppress cyclooxygenase-2 activity, inhibit angiogenesis, and decrease bone loss. As with other botanical substances, dried hops flowers contain very small amounts of alpha acids. Supercritical CO₂ extraction of dried hops cones can produce a thick, high viscosity resin, which can contain a high percentage of alpha acids, a very low percentage of beta acids, and almost no essential oils.

While supercritical CO₂ extraction is an effective means of providing alpha acids in a highly concentrated form, it would be useful to provide the primary constituents of hops extracts in other dosage formulations. Therefore, a method of converting hops extract resin into other forms and with different hops constituent profiles would be highly desirable.

### SUMMARY OF THE INVENTION

It has been recognized that it would be advantageous to be able to incorporate hops extract into oral dosage forms that do not cause nausea or other undesired side effects. It has also been recognized that patient compliance can be improved when such substances can be administered in a form that is better tolerated. In accordance with these recognitions, the present invention is drawn to a pharmaceutical composition, comprising a dry free flowing powder. The dry free flowing powder can include from 5 wt % to 85 wt % of a combination of alpha acids and iso-alpha acids, wherein the alpha acids and iso-alpha acids are each present at above 0.1 wt %, from 1 wt % to 50 wt % beta acids, and from 10 wt % to 90 wt % silica salt.

In an alternative embodiment, a pharmaceutical composition can comprise a dry free flowing powder including from 5 wt % to 80 wt % iso-alpha acids, from 1 wt % to 50 wt % beta acids, and from 10 wt % to 90 wt % silica salt. In this embodiment, the composition can be substantially free of alpha acids.

In another embodiment, a pharmaceutical composition can comprise a dry free flowing powder. The powder can include from 5 wt % to 85 wt % of a member selected from the group consisting of alpha acids, iso-alpha acids, and combinations thereof; from 1 wt % to 50 wt % beta acids; from 10 wt % to 90 wt % of an absorbing agent; and from 0.1 wt % to 10 wt % of an anti-oxidant.

In still another embodiment, a pharmaceutical composition can comprise a dry free flowing powder including from 5 wt % to 80 wt % iso-alpha acids, from 1 wt % to 50 wt % beta acids, and from 10 wt % to 90 wt % of an absorbing agent, and from 0.1 wt % to 10 wt % of an anti-oxidant. This composition can be substantially free of alpha acids.

In another embodiment, a method of increasing gastrointestinal tolerability can comprise steps of formulating a pharmaceutical composition including hops extract into a powdered oral dosage form, and orally delivering the dosage form to a subject at a dosage level for achieving a therapeutic effect. In this embodiment, the oral dosage form can be formulated for improvement in gastrointestinal tolerability as evidenced by improvement in at least 30% of subjects compared to delivering the same amount of hops extract in resin form.

In another embodiment, a method of preparing a dry free flowing pharmaceutical composition can comprise the steps of concentrating hops into a viscous liquid extract, and mixing the extract with silica salt to form the dry free flowing pharmaceutical composition.

### BRIEF DESCRIPTION OF THE DRAWINGS

FIG. 1 is a high performance liquid chromatogram (HPLC) of a purified and fractionated hops resin standard consisting of 95 wt % alpha acids;

FIG. 2 is an HPLC of a commercially available hops resin containing 80 wt % alpha acids, which is used fill a softgel capsule for tolerability testing of purified resin in humans; and

FIG. 3 depicts an HPLC of a powder having improved human tolerability which is produced from a 60% alpha acid resin, where the upper panel depicts the initial HPLC and the lower panel depicts the same HPLC after application of enhanced integrator software to make smaller peaks more visible.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

It is to be noted that, as used in this specification and the appended claims, the singular forms “a,” “an,” and “the” include plural referents unless the context clearly dictates otherwise.

As used herein, “administration,” and “administering” refer to the manner in which a drug, formulation, or composition is introduced into the body of a subject. Administration can be accomplished by various art-known routes such as oral, parenteral, transdermal, inhalation, implantation, etc. Thus, an oral administration can be achieved by swallowing, chewing, or sucking an oral dosage form comprising active agent(s). Parenteral administration can be achieved by injecting a composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well known in the art.

The terms “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of an active agent can achieve a therapeutic effect that is measurable by the subject receiving the active agent. The determination of an effective amount is well within the ordinary skill in the art of pharmaceutical, medicinal, and health sciences.

As used herein, “carrier” or “inert carrier” refers to typical compounds or compositions used to carry active ingredients, such as polymeric carriers, liquid carriers, or other carrier vehicles with which a bioactive agent, such as hops extract, may be combined to achieve a specific dosage form. As a generally principle, carriers do not substantially react with the bioactive agent in a manner which substantially degrades or otherwise adversely affects the bioactive agent or its therapeutic potential. In accordance with embodiments of the present invention, silica salt is admixed with hops extract to form a powder that can be delivered to subjects with increased tolerability. However, other carriers can be used in addition to the silica salt, or can be mixed or blended with the powder in accordance with embodiments of the present invention.

The term “high intensity mixer” refers to mixers capable of producing high shear rates during mixing. High intensity mixing can be achieved through a variety of combinations of mixing blade speed, mixing plow configuration, turnover volume, and shear. The various configurations and locations of the mixing elements and the resulting mechanical motion are designed to force the product into appropriate components of axial and radial motion, so as to thoroughly mix ingredients to form dry product without the use of a added solvent. For example, hops extract, which is a viscous, honey-like liquid, can be admixed with silica salt in such a high intensity mixer without any added solvent to form a dry free-flowing powder. High intensity mixers typically exhibit rotor tip speeds of over 40 feet per second.

The term “supercritical carbon dioxide” or “supercritical CO₂” refers to carbon dioxide gas that has been heated and pressurized until it is beyond its critical state, e.g., above 310° C. and 73 atmospheres. Gases in this state have been found to be excellent solvents, possessing a pressure-tunable dissolving power, liquid-like density, and gas-like transport properties. The term “supercritical carbon dioxide extraction” refers to a separation process using supercritical CO₂ as a solvent. To be clear, the supercritical CO₂ is used as a solvent to form hops extract. However, once the extract is formed, no solvent is necessary to conduct the powder conversion from liquid hops extract to its powder form in accordance with embodiments of the present invention.

The term “alpha acid(s)” refers to humulone, cohumulone, adhumulone, dihydrohumulone, dihydrocohumulone, dihydroadhumulone, or any mixture thereof. This term does not refer to the various isomers of these three compounds, however. Instead, the term “iso-alpha acid(s)” refers to iso-humulone, iso-cohumulone, iso-adhumulone, trans-iso-humulone, cis-iso-humulone, trans-iso-cohumulone, cis-iso-cohumulone, cis-iso-adhumulone, trans-iso-adhumulone, dihydro-iso-humulone, and dihydro-iso-adhumulone, or any mixture thereof.

The term “beta acid(s)” refers to lupulone, colupulone, adlupulone, prelupulone, postlupulone, or any mixture thereof.

As used herein, “subject” refers to an animal, such as a mammal, that may benefit from the administration of the compositions or formulations of the present invention. Most often, the subject will be a human.

The term “about” when referring to a numerical value or range is intended to encompass the values resulting from experimental error that can occur when taking measurements.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

In accordance with this, a dry, free flowing pharmaceutical powder composition can comprise alpha acids, iso-alpha acids, and/or beta acids admixed with silica salt in a solvent-free mixing process. In one embodiment, all three types of extracted acids can be present, and in another embodiment, alpha acids can be substantially converted to iso-alpha acids within the extract. Alpha acids may be useful in clinical applications where the suppression of cyclooxygenase-2 activity, inhibition of angiogenesis, or decreased bone loss is desired. In addition to the silica salt, the compositions of the present invention may also include other additives. The compositions of the present invention are different than those currently known for at least two reasons, both of which contribute to enhancing therapeutic effects when delivered to subjects. Specifically, the compositions of the present invention contain lower concentrations of alpha acids than are typically found in pure hops extract, and further, these compositions are in a powder form using highly absorptive silica salts as the powder conversion agent. These two modifications provide a desired therapeutic effect without the nausea that can accompany ingestion of pure hops extract. Further, the reduction of nausea, stomach gas, and other undesired side effects associated with the delivery of pure hops extract or softgel delivery of hops extract can increase subject compliance, thereby increasing the therapeutic effect.

The acids in the above embodiments can be obtained from the cone flower of the hops plant. Therefore, this invention is also drawn toward a method of producing a dry, free flowing pharmaceutical composition by mixing a resin of hops extract with silica salt. The hops extract may be obtained through supercritical CO₂ extraction of hops cones, or through other extraction techniques. Other additives, such as maltodextrin or ascorbic acid, may also be included to produce the final composition.

Pharmaceutically useful botanical compounds are often only present in low amounts in their respective plants. The cone flower of hops (Humulus lupulus L.) is no exception, and has been found to contain a number of agents with possible therapeutic applications. For example, dried hops flowers contain small amounts of alpha acids and beta acids. To collect a more concentrated form of the active ingredients in hops, these ingredients may be extracted and purified by various techniques, including organic solvent extraction, microwave extraction, or supercritical CO₂ extraction. The present invention may utilize hops extract collected by any of these techniques, or other techniques suitable for extracting plant materials.

In organic solvent extraction, hops cone flowers are steeped in a solvent such as ethanol or hexanes, allowing the essential oils and other soluble materials from the plant to dissolve. The resulting solution is then recovered and the solvent evaporated therefrom, often by either filtering the solution, heating it under a vacuum, or both. As many organic solvents are toxic, it is desirable to remove as much of the solvent as possible. In most instances, the remaining material takes on the consistency of a resin, an oil, or a pasty substance.

Hops may also be extracted using microwaves, where the hops cones are placed in a solvent and the mixture is then subjected to intense microwave radiation. The microwave radiation disrupts the physical structure of the plant material so that the desired compounds migrate directly into the solvent. Solvents that are transparent to microwaves are usually used, so that the solvent itself is not heated. The solvent is then separated from the resulting slurry by filtration, evaporation, or both.

Another method of extracting hops is supercritical carbon dioxide (CO₂) extraction. This process involves passing supercritical carbon dioxide gas through the hops and thereby dissolving the constituents into the gas. The gas is then recovered and depressurized, causing the dissolved materials to precipitate out. If desired, the recovered gas may be re-pressurized and recycled back through the hops for further extraction. One advantage of supercritical CO₂ extraction over solvent-based techniques is that substantially no solvent residues remain with the extract. The resulting product is a thick, high viscosity resin, oil, or other fluid like material, containing a very high percentage of alpha acids, very little beta acids, and essentially no essential oils. The hops resin produced by supercritical CO₂ extraction can contain 60 wt % or more alpha acids, compared to 5 wt % to 15 wt % typically present in extracts from organic solvents, and 1-5% present in dried hops cones. The extract may then be fractionated so as to achieve a resin containing over 80% alpha acids. Supercritical CO₂ extraction therefore yields a resin that is much more potent than dried hops flowers.

Regardless of the extraction method used, one skilled in the art would have thought to incorporate hops extracts into soft gelatin (softgel) capsules as a viscous liquid, e.g., combined with an oil such as soy bean oil or olive oil. However, it has been discovered herein that patient or subject compliance issues hamper the pharmaceutical use of more pure forms of hops resin, including administration in high viscosity liquid forms. Due to low native levels of pharmaceutically useful substances, large doses are often used in order to achieve a therapeutic effect when administering nutraceuticals, essential oils, and/or botanical substances. However, in order to enable good patient compliance with these treatments, it is necessary to administer the least number of dosage forms per day. Therefore, a higher concentration of active agent per dose would be desirable. Though relatively pure forms of hops resin can be prepared, trials show that these forms are not tolerated well by patients. For example, a supercritical CO₂ extract of hops containing 80 wt % alpha acids was encapsulated into soft gelatin capsules with olive oil. These capsules contained 150 mg of alpha acids, and a two-capsule dose therefore contained 300 mg of alpha acids. Ten individuals were given a single dose of two capsules. Over 50% of the subjects complained of various degrees of nausea or gastrointestinal side effects. These effects are believed to be related, in part, to difficulty in digesting a bolus of concentrated resin. Generally, pharmaceutical compositions are multi-particle formations that rapidly disintegrate into a large number of subunits when ingested. Thus, it has been recognized that patient tolerance of hops extracts could be increased by administering of the hops extract in powder form. It was also discovered that the tolerance of hops extract could also be improved by administering hops acids in weight percentages different from those found in more pure hops resin.

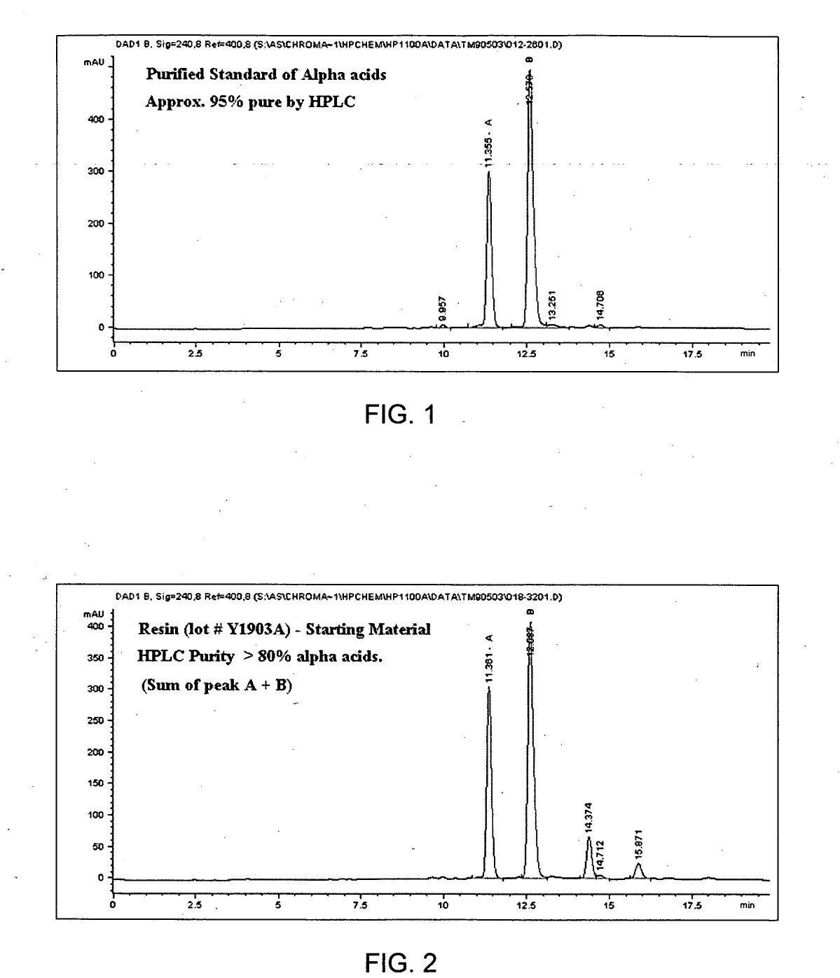

In light of these recognitions, the present invention provides a pharmaceutical composition derived from hops resin. In each embodiment, the composition comprises a dry, free flowing powder obtained by mixing various concentrations of hops resin with a silica salt. In each embodiment, the composition contains hops acids in different weight percentages than are found in pure hops resin, as shown by high performance liquid chromatography (HPLC) analysis. For example, a typical HPLC of a purified hops fraction consisting of 95 wt % alpha acids is shown in FIG. 1. The two peaks (at 11.355 and 12.570 minutes) correspond to the alpha acids humulone and cohumulone, respectively. FIG. 2 shows an HPLC of a less purified fractioned hops resin which contains about 80 wt % of the same alpha acids (indicated by the large peaks at 11.361 and 12.587 minutes) as well as a small amount of beta acids lupulone and colupulone (indicated by the two smaller peaks at 14.374 and 15.871). These two compositions, when administered in softgel capsules, can produce nausea and other gastrointestinal side effects in humans.

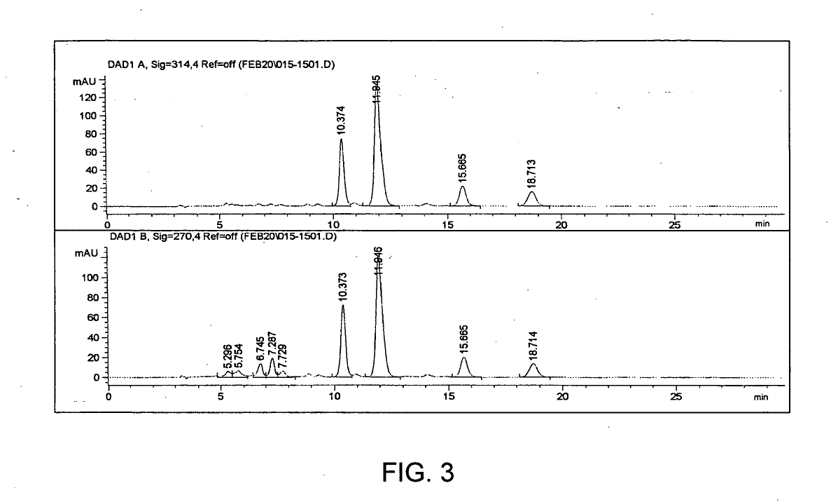

In accordance with the present invention, FIG. 3 depicts a composition in accordance with embodiments of the present invention. The upper panel of FIG. 3 shows a typical HPLC analysis of a powder (60 wt % alpha acids) prepared according to an embodiment of the present invention from hops extract, where the finished powder also contains calcium silicate and maltodextrin. The extract was obtained by supercritical CO₂ extraction. The peaks reveal a different analytical signature in the powdered composition than in the hops resins. The peaks at 10.374 and 11.945 represent the alpha acids cohumulone and adhumulone, respectively, while the two peaks on the right (at 15.665 and 18.713) represent the beta acids colupulone and lupulone, respectively. This same spectrum is shown in the lower panel utilizing enhanced integrator software that reveals smaller peaks with more detail. The smaller peaks on the left, which correspond to iso-alpha acids (isohumulone, isocohumulone, and isoadhumulone), reveal the relative percentages of the iso-alpha acids.

The unique and unexpected analytical signature seen in the powder prepared in accordance with embodiments of the present invention has also been accompanied by an increased tolerance in patients for oral administration. As mentioned above, of 10 subjects who were given two softgel capsules of supercritical CO₂ extract of hops containing 300 mg of 80 wt % alpha acids, 50 % complained of gastrointestinal distress. After a two-week wash out period, the same subjects were given two 500 mg tablets of a powder containing 30 wt % alpha acids (300 mg alpha acids) in a composition of the instant invention, and none experienced any of the side effects of the softgel capsules containing the pure resin in olive oil, even on an empty stomach.

In accordance with these recognitions, in one embodiment of the present invention, a pharmaceutical composition is provided comprising from 5 wt % to 85 wt % a combination of alpha acids and iso-alpha acids, from 1 wt % to 50 wt % beta acids, and 10 wt % to 90 wt % silica salt. In another embodiment of the invention, the alpha acids are present at a greater weight percentage than beta acids. In more specific embodiments, the alpha acids can be present in the final powder composition at from 20 wt % to 40 wt % (or from 25 wt % to 35 wt %), and/or the beta acids can be present at from 5 wt % to 12 wt % (or from 8 wt % to 12 wt %). The silica salt can optionally be present in the final powder composition at from 15 wt % to 50 wt % (or 20 wt % to 40 wt %).

In another embodiment, the pharmaceutical composition comprises from 0.1 wt % to 40 wt % iso-alpha acids, from 1 wt % to 50 wt % beta acids, and from 10 wt % to 90 wt % silica salt. In this embodiment, the composition can be substantially free of alpha acids. In one embodiment, the the iso-alpha acids can be present at from 20 wt % to 40 wt % (or from 25 wt % to 35 wt %), and/or the beta acids can be present at from 5 wt % to 12 wt % (or from 8 wt % to 12 wt %). The silica salt can optionally be present in the final powder composition at from 15 wt % to 50 wt % (or 20 wt % to 40 wt %).

In another embodiment, a pharmaceutical composition can comprise a dry free flowing powder. The powder can include from 5 wt % to 85 wt % of a member selected from the group consisting of alpha acids, iso-alpha acids, and combinations thereof; from 1 wt % to 50 wt % beta acids; from 10 wt % to 90 wt % of an absorbing agent; and from 0.1 wt % to 10 wt % of an anti-oxidant. In more specific embodiments, the alpha acids can be present in the final powder composition at from 20 wt % to 40 wt % (or from 25 wt % to 35 wt %), and/or the beta acids can be present at from 5 wt % to 12 wt % (or from 8 wt % to 12 wt %). The silica salt can optionally be present in the final powder composition at from 15 wt % to 50 wt % (or 20 wt % to 40 wt %).

In still another embodiment, a pharmaceutical composition can comprise a dry free flowing powder including from 5 wt % to 80 wt % iso-alpha acids, from 1 wt % to 50 wt % beta acids, and from 10 wt % to 90 wt % of an absorbing agent, and from 0.1 wt % to 10 wt % of an anti-oxidant. This composition can be substantially free of alpha acids. In one embodiment, the the iso-alpha acids can be present at from 20 wt % to 40 wt % (or from 25 wt % to 35 wt %), and/or the beta acids can be present at from 5 wt % to 12 wt % (or from 8 wt % to 12 wt %). The silica salt can optionally be present in the final powder composition at from 15 wt % to 50 wt % (or 20 wt % to 40 wt %).

Any these compositions can be prepared by concentrating hops into a viscous liquid extract, and mixing the extract with an absorbing agent, such as silica salt, to form the dry free flowing pharmaceutical composition. Other absorbing agents that can be used alone or in combination with silica salt include carbohydrates, proteinaceous materials, fibers, silica, and combinations thereof. In one embodiment, the step of mixing includes using a high intensity mixer capable of generating high shear rates equivalent to rotor tip speeds greater than 40 feet per second.

In embodiments that utilize silica salt, though any silica salt can be used that is functional in accordance with embodiments of the present invention, in one embodiment, a preferred silica salt that can be used is calcium silicate. Other silica salts that can be used include magnesium silicate, sodium silicate, and potassium silicate, for example. The oil absorption of silica materials is about 200-500 ml/100 g, and the bulk specific gravity is about 0.1 g/ml or less. Precipitated amorphous calcium silicate is available from various suppliers. For example, Hubersorb® 600 is available from J. M. Huber Corp., Havre de Grace, Md. Hubersorb® calcium silicate has an oil absorption capacity of 475 cc/100 g, a BET surface area of 300 m²/g, and contains about 19 wt % calcium.

In embodiments that utilize anti-oxidants, as alpha acids are particularly susceptible to oxidation during processing, the use of anti-oxidant can improve the retention of the alpha acids or iso-alpha acids in desired ratios. In other words, the presence of the anti-oxidant can be used for not only a therapeutic effect, can also affect the amount of alpha acids or iso-alpha acids in the final composition. In one embodiment, ascorbic acid (e.g., 0.5 wt % to 10 wt %) may be optionally included to prevent the oxidation of the alpha acids. In other embodiments, the anti-oxidant can include a member selected from the group consisting of tocopherols, retinal, tocotrienols, carotenoids, catechins, indoles, isoflavones, phenols, phytoestrogen, polyphenols, saponins, selenium, glutathione, lipoic acid, superoxide dismutase (SOD), glutathione peroxidase, glutathione reductase, iron, copper, zinc, manganese, ferritin, lactoferrin, albumin, ceruloplasmin, carnosol, coumarins, dithiothiones, monoterpenes, quercetin, resveratrol, and mixtures thereof.

Additional materials may be added at from 0.1 wt % to 50 wt %, such as absorbent carriers and/or fillers, which include, but are not limited to carbohydrates, proteinaceous materials, fibers, silica. Specific examples of these and other materials that can be included are maltodextrin (e.g., 5 wt % to 30 wt %), corn starch, corn syrup solids, glucose, sodium caseinate, casein, soy protein isolate, whey protein, acacia gum, guar gum, cellulose, carboxymethylcellulose, and pectin.

Additionally, a variety of additives or carriers can be incorporated into the compositions for their intended functions. These additives are usually used in small amounts, but if used as absorbing agents, they may be used in larger amounts. Examples of classes of additives include, but are not limited to, vitamins, organic acids, free amino acids, amino acid salts, excipients, lubricants, hydrocolloid suspending agents, buffering agents, disintegrating agents, stabilizers, foaming agents, pigments, coloring agents, fillers, bulking agents, sweetening agents, flavoring agents, fragrances, release modifiers,flow control agents, hydroscopicity minimizing agents, pH control agents, catalysts, , dust control agents, binders, taste-reducing agents, capsule shells, shellacs, waxes, emulsifiers, oils, combinations thereof, and other known additives.

As mentioned previously, there are certain advantages to delivering a free-forming powder rather than a high viscosity botanical extract to a subject. However, there are additional advantages of generating a powder other than those described previously. For example, a free flowing powder can be weighed more precisely, and can be used in machinery to manufacture pharmaceutical dosage forms without clogging the apparatus or plugging various portals. In the pharmaceutical industry, many therapeutic agents are high viscosity fluids, and would need to be converted to a powder to enable a proper dosage form such as a tablet or a capsule. Once a high viscosity fluid is converted into a powder, it can then be further processed into many different dosage forms such as tablets, two piece hard shell gelatin capsules, or powders that can be reconstituted in liquids or added to foods or confections.

Spray drying is one of the most common methods for creating powder from fluids. Spray drying has the limitation of low yields, and as high viscosity fluids often have a honey-like consistency, these fluids typically must be solubilized or diluted with a solvent to enable the fluid to be sprayed without clogging the spray nozzles. The resulting powders are usually significantly diluted to below 20 wt % of the original high viscosity fluid, and are frequently diluted to from 5 wt % to 10 wt %. Spray drying is usually accomplished by dissolving maltodextrin in water and dissolving the therapeutic agent into the same solution and spray drying until a powder is produced, usually with about a 5 wt % moisture level. For example, a 30 wt % solution of maltodextrin in water is prepared for dissolving the therapeutic agent. Usually after spray drying, a 10 wt % yield results, meaning there was a 90 wt % dilution. Spray drying of hops extracts in this manner typically results in no greater than 10 wt % of the starting level of alpha acids that were in the original extract. This level of alpha acids is comparable to that found in powdered hops leaves before extraction (5 wt % to 10 wt %). Therefore, it is not practical to spray dry a botanical extract such as hops for pharmaceutical or therapeutic purposes, because there is a significant loss in potency.

Fluid bed granulation, agglomeration, or coating is also one of the most common techniques used at the present time for production of powders. The core drug is first preheated in the vessel to about 30° C. with hot air, placing the particles in suspension. The floating particles are then sprayed with an aqueous suspension to provide a coating, while drying at the same time. Fluid bed equipment is available as “top spray,” “bottom spray,” and “tangential-spray,” depending whether the spray nozzles is sprayed into the vessel from the top, bottom, or the side. Inlet temperature, spray rate, and air throughput must be adjusted to provide an optimal end product. Furthermore, the finished particles must be subjected to a post-drying period at around 40° C., where any residual moisture can be driven off. In some case, this last drying period may be up to 24 hours. In this case, the drug or therapeutic agent is typically already be in the form of a powder. Traditional spray drying, fluid bed drying, or agglomeration involve the use of aqueous solutions or organic solvents in preparing the powder.

Many of the polymers that could be used to make powders in the fluid bed process require solvents such as acetone, isopropyl alcohol, chlorinated solvents, alkanes, methyl ethyl ketone, cyclohexane, toluene, carbon tetrachloride, chloroform, and the like. Evaporation of these solvents becomes an environmental concern, and in many states, it is illegal to release the resulting emissions into the atmosphere. Aqueous polymers are limited mainly to ethyl cellulose and methacrylic acid esters such as polymethacrylate dispersions. In addition, 10 wt % to 20 wt % of a suitable plasticizer such as triethyl citrate must be added to the polymer. Typical aqueous ethyl cellulose polymers currently in wide use include Surelease®, (Colorcon, West Point, Pa.), and Aquacoat®, (FMC Corporation, Philadelphia, Pa.). The recommended procedure for manufacturing sustained-release compositions using Aquacoat® requires curing in a tray dryer at 60° C. for at least two hours to insure reproducible release profiles. Subjecting drugs and other therapeutic compounds such as botanical extracts to 60° C. temperatures for 2 hours or more is likely to result in a loss of potency or degradation of active principles, and is especially problematic for substances with low melting points. Botanical extracts, in particular, have many volatile compounds that can be destroyed if kept at high temperatures for long periods.

Another method for creating dry pharmaceutical compositions is wet granulation. Wet granulation involves mixing the drug or therapeutic agent with water in a conventional high-speed mixer until a pasty mass, and then dried in an oven over 24 hours at 60° C. Wet granulations have the additional draw back in that they can affect the potency of botanical extracts by causing instability, or transformation. In addition, when dried at 60° C., many sensitive active principles are lost.

Another method of producing powders is by starting with sugar spheres or nonpareils. The sugar serves as a seed for the creation of a particle. The sugar spheres are also processed in a fluid bed granulator, but the drug must be dissolved in an aqueous solution and sprayed onto the sugar spheres, followed by spray coating with polymers that produce sustained release as previously mentioned. This system results in large particles that are not acceptable in most drink mix applications, and botanical extracts cannot be dissolved enough to use in this system. The therapeutic agent is absorbed into the sugar particle. The smallest starting particle size for nonpareils is about 60 mesh (US standard sieve number). After coating, the particles are often 30 mesh and larger. The large particle size also makes encapsulation or tableting more difficult.

Though several exemplary methods of preparing powders are described herein as being less desirable for one reason or another, it should be emphasized that to the extent that any of these methods can be used to prepare the compositions of the present invention, they are included as viable methods. This being stated, it has been discovered that by converting high viscosity fluid to powder by high shear mixing in a solvent-free environment, a free flowing powder can be formed that has higher concentrations of alpha acids and/or iso-alpha acids, depending on the particular embodiment.

In accordance with this recognition, a preferred method for making a powder is described herein which relates to the use of high shear and/or high intensity mixing, preferably without the use of any added solvent (other than that which may residually be present in the extract that remains due to the extraction process itself). Mixing technology has many subtleties, such as degrees of speed, precision, efficiency, configuration of mixing plows, turnover volume, and shear. High turnover mixing and high shear mixing are facilitated by high intensity mixers. In contrast to a fluid bed granulator, where the powder particles are suspended by high-pressurized air itself (air suspension), in a high intensity and/or high shear mixer, the powder particles are suspended (fluidized) by mechanical motion.

The present invention is directed toward a method of producing dry, free-flowing compositions by mixing hops extract resin with suitable absorbent carriers in an appropriate mixing vessel to form a dry powder. Water or other solvents are preferably not used at all for the mixing step. Thus, the compositions that are formed are free flowing, and suitable for tableting or filling into two-piece capsules. Other useful dosage forms that may be made from such powders include beads, granules, aggregates, powder, solids, semi-solids, or suspensions. Further, lotions, as well as transdermal delivery systems including dermal patches, implantable forms or devices, nasal mists, suppositories, salves, and ointments can also be useful forms of delivery. Cosmetic powders may also be produced.

The apparatus used to manufacture the powders of the present invention is exemplified by a Littleford vertical or horizontal high intensity mixer (Littleford Day Inc., Florence, Ky.), or a comparable high intensity mixer or plow mixer. The unique mixing action of the auger shaft or plows (blades) revolving at a high rate of speed causes the particles to fluidize in free space, providing a high volume rate of material transfer throughout the entire length of the vessel. This results in the mixing, blending and adsorption, and reacting of the hops resin onto the unique compositions that will be described. In addition, the vessel can be fitted with high speed impact choppers. After processing this way, the material is discharged as a free-flowing powder.

One example of a high intensity mixer, the Littleford W-10 laboratory mixer, has a 0.25 cubic foot working capacity, stainless steel construction, and a 3 HP, 1730 rpm variable speed drive motor. The mixing unit is jacketed to enable passage of hot water or steam around the vessel to elevate the temperature of the internal contents if desired. The mixing blades inside the vessel are capable of high RPM, and are typically operated at 1,000 to 3,000 RPM. High intensity mixing produces fluidized bed mixing action, assuring absolute axial and radial mixing. Larger high intensity mixers are available with capacities that enable scale up to commercial production batches. An ideal intermediate sized mixer of this type is the Littleford FM-130, which has a 130 liter (4.6 cubic feet), or 34 gallon capacity. Larger mixers of this type are available up to 25,000 liter capacity. The preparation of commercial batches of 1,000 kilos or more are possible in larger units.

In one embodiment, hops resin, silica salt, and optionally other additives are mixed in a jacketed high intensity mixer under high shear and speed with significant turn over until the high viscosity fluid is dried into a powder by admixture with the carriers. In another embodiment, the above ingredients are mixed while hot water or steam is passed through the jacket of the mixer. Elevated temperature promotes the isomerization of alpha acids, so the resulting composition may comprise a substantial weight percentage of iso-alpha acids while being substantially free of alpha acids.

The compositions of the present invention have great versatility in their application. The compositions can be used for treating various cancers, inflammatory conditions, as antibiotics, as anti-fungals, as anti-viral agents, for treatment of protozoa or various food born pathogens, and essentially any disease or malady for which hops has been found to ameliorate.

In a further embodiment, the compositions described herein can be formulated such that gastrointestinal tolerability is increased. In accordance with this, a method of increasing gastrointestinal tolerability can comprise steps of formulating a pharmaceutical composition including hops extract into a powdered oral dosage form, and orally delivering the dosage form to a subject at a dosage level for achieving a therapeutic effect. In this embodiment, the oral dosage form can be formulated for improvement in gastrointestinal tolerability as evidenced by improvement in at least 40% of subjects compared to delivering the same amount of hops extract in resin form.

In accordance with this embodiment, it was discovered that the pharmaceutical administration of pure hops resins administered with a liquid carrier, e.g., olive oil, etc., in a softgel tablet provided a low tolerability in many human subjects. These types of compositions, e.g., nutraceuticals, essential oils, and botanicals, are often delivered at relatively high dosages in order to achieve a therapeutic effect. Thus, more specifically, at these higher dosages which are desirable for achieving therapeutic effects in humans, it was discovered that the administration of highly concentrated hops resin in softgel capsules produced nausea in from 50% to 75% of subjects, which is believed to be related to the bolus of administered resin that is not easily assimilated in the digestive tract. Because of the nausea and other gastrointestinal side effects, issues of patient compliance are a concern with these types of formulations. Thus, it has been recognized that by providing hops extract in the form of a multi-particulate or powder, the composition is more easily dispersed within the gastrointestinal tract, thereby decreasing undesirable gastrointestinal side effects. Further, by manipulating the concentration of alpha acids, beta acids, and iso-alpha acids as described herein, acceptable therapeutic affect and reduced side effects can be balanced.

### EXAMPLES

### Example 1

### Preparation of a Powder from Hops Extract

Hops cones were extracted with supercritical CO₂, yielding a hops resin containing 60 wt % alpha acids. The hops resin was placed, with other ingredients, into a mixing vessel of a jacketed high intensity mixer according to the following proportions: 53.5 wt % hops resin, 25 wt % calcium silicate, and 21.5 wt % maltodextrin. The mixer was started and allowed to run until the hops resin and adsorbing agents formed a powder. This process was typically completed in about 10 to 20 minutes of mixing. The resulting powder was then discharged from the mixing vessel, and optionally screened. No solvent was added to the mixer during the mixing process. The final product was a very fine, free-flowing powder with a faint yellowish color. The powder was analyzed by HPLC, using methanol as an eluent, ICE-2 as a standard for alpha and beta acids, and DCHA as a standard for iso-alpha acids. The results of that analysis are shown below in Table 1.

TABLE 1

| | Weight |
|---|---|
| Constituent | percentage |
| Alpha acids | 30.9 |
| Beta acids | 10 |
| Iso-alpha acids | 4.3 |

This result shows that some of the alpha acids were converted into iso-alpha acids by this process. The resulting powder contained 35.2 wt % total alpha and iso-alpha acids. This yield is higher in alpha acid content than is present in powdered hops cones, an organic solvent based extract, or a supercritical CO₂ extract that has been spray dried.

### Example 2

### Preparation of a Hops Extract Powder with Higher Iso-alpha Acid Content

A hops resin containing 60 wt % alpha acids was converted into a powder using the same method and composition as described in Example 1, except that the contents of the mixing vessel were heated to 150° F. by running hot water or steam in the jacket of the mixer while mixing. The powder was analyzed by HPLC, using methanol as an eluent, ICE-2 as a standard for alpha and beta acids, and DCHA as a standard for iso-alpha acids. The results of the analysis are shown below in Table 2.

TABLE 2

| | Weight |
|---|---|
| Constituent | percentage |
| Alpha acids | 0.0 |
| Beta acids | 8 |
| Iso-alpha acids | 30 |

This result shows that heating the mixture to 150° F. resulted in all of the alpha acids being converted into iso-alpha acids. Some of the alpha acids may have been lost during the heating process, or may have been converted to alpha acid derivates.

### Example 3

### Preparation of a Hops Extract Powder with Ascorbic Acid

A hops resin containing 60 wt % alpha acids was converted into a powder using the same method as in Example 1, except that the following ingredients and proportions were used: 68 wt % hops resin, 25 wt % calcium silicate, 5.5 wt % maltodextrin, and 1.5 wt % ascorbic acid. HPLC analysis of the resulting powder yielded the results shown in Table 3 below:

TABLE 3

| | Weight |
|---|---|
| Constituent | percentage |
| Alpha acids | 31.9 |
| Beta acids | 11.9 |
| Iso-alpha acids | 8.8 |

This is an example of a hops extract powder containing a similar percentage of alpha acids as the powder in Example 1, with the addition of ascorbic acid to prevent oxidation of the alpha acids.

While the invention has been described with reference to certain preferred or illustrative embodiments, those skilled in the art will appreciate that various substitutions, modifications, changes, or omissions can be made without departing from the spirit of the invention. It is therefore intended that the invention be limited only by the scope of the appended claims.

1. A pharmaceutical composition, comprising a dry free flowing powder, comprising:
(a) from 5 wt % to 85 wt % of a combination of alpha acids and iso-alpha acids, wherein the alpha acids and iso-alpha acids are each present at above 0.1 wt %;
(b) from 1 wt % to 50 wt % beta acids; and
(c) from 10 wt % to 90 wt % silica salt.

2. A composition as in claim 1, with the proviso that the alpha acids are present at a greater weight percentage than the beta acids.

3. A composition as in claim 1, wherein the silica salt is calcium silicate.

4. A composition as in claim 1, further comprising an absorbent carrier selected from the group consisting of carbohydrates, proteinaceous materials, fibers, silica, and combinations thereof.

5. A composition as in claim 4, wherein carbohydrates are selected from the group consisting of maltodextrin, corn starch, corn syrup solids, and glucose.

6. A composition as in claim 5, wherein maltodextrin is present at 5 wt % to 30 wt %.

7. A composition as in claim 4, wherein the proteinaceous materials are selected from the group consisting of sodium caseinate, casein, soy protein isolate, and whey protein.

8. A composition as in claim 4, wherein the fibers are selected from the group consisting of acacia gum, guar gum, cellulose, carboxymethylcellulose, and pectin.

9. A composition as in claim 1, further comprising from 0.5 wt % to 10 wt % ascorbic acid.

10. A composition as in claim 1, wherein the alpha acids are present at from 20 wt % to 40 wt %.

11. A composition as in claim 10, wherein the alpha acids are present at from 25 wt % to 35 wt %.

12. A composition as in claim 1, wherein the beta acids are present at from 5 wt % to 12 wt %.

13. A composition as in claim 12, wherein the beta acids are present at from 8 wt % to 12 wt %.

14. A composition as in claim 1, wherein the silica salt is present at from 15 wt % to 50 wt %.

15. A composition as in claim 14, wherein the silica salt is present at from 20 wt % to 40 wt %.

16. A composition as in claim 1, wherein the alpha acids include humalone, cohumalone, adhumalone, or mixtures thereof.

17. A composition as in claim 1, wherein the beta acids include lupulone, colupulone, adlupulone, or mixtures thereof.

18. A composition as in claim 1, further comprising from 0.1 to 40 wt % iso-alpha acids, with the proviso that the iso-alpha acids are present at a smaller weight percentage in the composition than the alpha acids.

19. A composition as in claim 1, wherein the dry free flowing powder is compressed into a tablet.

20. A composition as in claim 1, wherein the dry free flowing powder is present in a capsule.

21. A pharmaceutical composition, comprising a dry free flowing powder, comprising:
(a) from 5 wt % to 80 wt % iso-alpha acids;
(b) from 1 wt % to 50 wt % beta acids; and
(c) from 10 wt % to 90 wt % silica salt,
said composition being substantially free of alpha acids.

22. A composition as in claim 21, with the proviso that the iso-alpha acids are present at a greater weight percentage than the beta acids.

23. A composition as in claim 21, wherein said substantially free is less than 0.1 wt % alpha acids.

24. A composition as in claim 21, wherein the silica salt is calcium silicate.

25. A composition as in claim 21, further comprising an absorbent carrier selected from the group consisting of carbohydrates, proteinaceous materials, fibers, silica, and combinations thereof.

26. A composition as in claim 25, wherein carbohydrates are selected from the group consisting of maltodextrin, corn starch, corn syrup solids, and glucose.

27. A composition as in claim 26, wherein maltodextrin is present at 5 wt % to 30 wt %.

28. A composition as in claim 25, wherein the proteinaceous materials are selected from the group consisting of sodium caseinate, casein, soy protein isolate, and whey protein.

29. A composition as in claim 25, wherein the fibers are selected from the group consisting of acacia gum, guar gum, cellulose, carboxymethylcellulose, and pectin.

30. A composition as in claim 21, further comprising from 0.5 wt % to 10 wt % ascorbic acid.

31. A composition as in claim 21, wherein the iso-alpha acids are present from 20 wt % to 40 wt %.

32. A composition as in claim 31, wherein the iso-alpha acids are present from 25 wt % to 35 wt %.

33. A composition as in claim 21, wherein the beta acids are present at from 5 wt % to 12 wt %.

34. A composition as in claim 33, wherein the beta acids are present at from 8 wt % to 12 wt %.

35. A composition as in claim 21, wherein the silica salt is present at from 15 wt % to 50 wt %.

36. A composition as in claim 35, wherein the silica salt is present at from 20 wt % to 40 wt %.

37. A composition as in claim 21, wherein the iso-alpha acids include isohumalone, isocohumalone, isoadhumalone, or mixtures thereof.

38. A method of preparing a dry free flowing pharmaceutical composition, comprising:
(a) concentrating hops into a viscous liquid extract; and
(b) mixing the extract with silica salt to form the dry free flowing pharmaceutical composition.

39. A method as in claim 38, wherein the dry free flowing pharmaceutical composition includes:
(a) from 5 wt % to 85 wt % of a combination of alpha acids and iso-alpha acids, wherein the alpha acids and iso-alpha acids are each present at above 0.1 wt %;
(b) from 1 wt % to 50 wt % beta acids; and
(c) from 10 wt % to 90 wt % silica salt.

40. A method as in claim 38, wherein the dry free flowing pharmaceutical composition includes:
(a) from 5 wt % to 80 wt % iso-alpha acids;
(b) from 1 wt % to 50 wt % beta acids; and
(c) from 10 wt % to 90 wt % silica salt,
said composition being substantially free of alpha acids.

41. A method as in claim 38, wherein the step of mixing includes mixing using a high intensity mixer capable of generating high shear rates or rotor blade tip speeds greater than 40 feet per second.

42. A method as in claim 38, further comprising addition of an absorbent carrier selected from the group consisting of carbohydrates, proteinaceous materials, fibers, silica, and combinations thereof.

43. A method as in claim 38, further comprising the addition of an anti-oxidant.

44. A pharmaceutical composition, comprising a dry free flowing powder, comprising:
(a) from 5 wt % to 85 wt % of a member selected from the group consisting of alpha acids, iso-alpha acids, and combinations thereof;
(b) from 1 wt % to 50 wt % beta acids;
(c) from 10 wt % to 90 wt % of an absorbing agent; and
(d) from 0.1 wt % to 10 wt % of an anti-oxidant.

45. A composition as in claim 44, wherein the anti-oxidant includes a member selected from the group consisting of tocopherols, retinal, tocotrienols, carotenoids, catechins, indoles, isoflavones, phenols, phytoestrogen, polyphenols, saponins, selenium, glutathione, lipoic acid, superoxide dismutase (SOD), glutathione peroxidase, glutathione reductase, iron, copper, zinc, manganese, ferritin, lactoferrin, albumin, ceruloplasmin, carnosol, coumarins, dithiothiones, monoterpenes, quercetin, resveratrol, and mixtures thereof.

46. A composition as in claim 44, wherein the anti-oxidant is ascorbic acid.

47. A composition as in claim 44, wherein the alpha acids and iso-alpha acids are each present at above 0.1 wt %.

48. A composition as in claim 44, wherein the absorbing agent is selected from the group consisting of carbohydrates, proteinaceous materials, fibers, silica, silica salts, and combinations thereof.

49. A composition as in claim 45, wherein the absorbing agent is the silica salt.

50. A composition as in claim 49, wherein the silica salt is calcium silicate.

51. A composition as in claim 44, wherein the alpha acids are present at from 20 wt % to 40 wt %.

52. A composition as in claim 51, wherein the alpha acids are present at from 25 wt % to 35 wt %.

53. A composition as in claim 44, wherein the beta acids are present at from 5 wt % to 12 wt %.

54. A composition as in claim 53, wherein the beta acids are present at from 8 wt % to 12 wt %.

55. A composition as in claim 49, wherein the silica salt is present at from 15 wt % to 50 wt %.

56. A composition as in claim 55, wherein the silica salt is present at from 20 wt % to 40 wt %.

57. A pharmaceutical composition, comprising a dry free flowing powder, comprising:
(a) from 5 wt % to 80 wt % iso-alpha acids;
(b) from 1 wt % to 50 wt % beta acids;
(c) from 10 wt % to 90 wt % of an absorbing agent; and
(d) from 0.1 wt % to 10 wt % of an anti-oxidant,
said composition being substantially free of alpha acids.

58. A composition as in claim 57, with the proviso that the iso-alpha acids are present at a greater weight percentage than the beta acids.

59. A composition as in claim 57, wherein said substantially free is less than 0.1 wt % alpha acids.

60. A composition as in claim 57 wherein the anti-oxidant includes a member selected from the group consisting of tocopherols, retinal, tocotrienols, carotenoids, catechins, indoles, isoflavones, phenols, phytoestrogen, polyphenols, saponins, selenium, glutathione, lipoic acid, superoxide dismutase (SOD), glutathione peroxidase, glutathione reductase, iron, copper, zinc, manganese, ferritin, lactoferrin, albumin, ceruloplasmin, camosol, coumarins, dithiothiones, monoterpenes, quercetin, resveratrol, and mixtures thereof.

61. A composition as in claim 57, wherein the anti-oxidant is ascorbic acid.

62. A composition as in claim 57, wherein the absorbing agent is selected from the group consisting of carbohydrates, proteinaceous materials, fibers, silica, silica salts, and combinations thereof.

63. A composition as in claim 62, wherein the absorbing agent is the silica salt.

64. A composition as in claim 63, wherein the silica salt is calcium silicate.

65. A composition as in claim 57, wherein the iso-alpha acids are present from 20 wt % to 40 wt %.

66. A composition as in claim 65, wherein the iso-alpha acids are present from 25 wt % to 35 wt %.

67. A composition as in claim 57, wherein the beta acids are present at from 5 wt % to 12 wt %.

68. A composition as in claim 67, wherein the beta acids are present at from 8 wt % to 12 wt %.

69. A composition as in claim 63, wherein the silica salt is present at from 15 wt % to 50 wt %.

70. A composition as in claim 69, wherein the silica salt is present at from 20 wt % to 40 wt %.

71. A method of increasing gastrointestinal tolerability, comprising:
(a) formulating a pharmaceutical composition including hops extract into a powdered oral dosage form;
(b) orally delivering the dosage form to a subject at a dosage level for achieving a therapeutic effect, said oral dosage form providing improved gastrointestinal tolerability in at least 40% of subjects compared to delivering the same amount of hops extract in resin form.

72. A method as in claim 71, wherein the pharmaceutical composition includes:
(a) from 5 wt % to 85 wt % of a member selected from the group consisting of alpha acids, iso-alpha acids, and combinations thereof;
(b) from 1 wt % to 50 wt % beta acids; and
(c) from 10 wt % to 90 wt % of an absorbing agent.

73. A method as in claim 72, wherein the absorbing agent is a silica salt.

74. A method as in claim 73, wherein the silica salt is calcium silicate.

75. A method as in claim 72, further comprising from 0.1 wt % to 10 wt % of an anti-oxidant.

76. A method as in claim 75, wherein the anti-oxidant is ascorbic acid.

77. A method as in claim 72, wherein the alpha acids and the iso-alpha acids are each present at least at 0.1 w %.

78. A method as in claim 72, wherein the composition is substantially free of alpha acids.

79. A method as in claim 71, wherein the powdered oral dosage form is compressed into a tablet.

80. A method as in claim 71, wherein the powdered oral dosage form is present in a capsule.
